(19) United States
(12) Patent Application Publication
(10) Pub. No.: US 2006/0157059 A1
(43) Pub. Date: Jul. 20, 2006

(54) End lighted endotracheal tube

(76) Inventors: Douglas R. Johnson, Mission Viejo, CA (US); Geoffrey Garth, Long Beach, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 11/039,186

(22) Filed: Jan. 19, 2005

(51) Int. Cl. A62B 9/06; A61M 16/00

(52) U.S. Cl. 128/207.14; 128/207.15; 128/200.26

(57) ABSTRACT

An endotracheal tube has a light-emitting diode or other low temperature, built-in light source forward of the cuff.

### FIELD OF INVNENTION

The field of the invention is medical devices, and more particularly endotracheal tubes.

### BACKGROUND

Endotracheal tubes are commonplace in the medical field, both in medical facilities and in the field. Despite the prevalence of such tubes, however, their insertion remains a matter of training and skill.

The advent of lighted endotracheal tubes significantly improved the success rate of intubation. Lighted stylet guided intubation has been found to be particularly useful for difficult situations, such as anterior larynx scarring and bloody airway, and is in any event generally less traumatic for the patient. Among other things, lighted stylet intubation reduces manipulation of the head and neck, and the need for excessive opening of the mouth. There are numerous such devices on the market, including Trachlite® (Rusch), Trachlight® (Laerdal), Surch-lite® (Aaron Medical), and Lightwand”®.

Endotracheal tubes can also be lighted without a removable stylet. For example, it is known to provide a light source exterior to the endotracheal tube, and run the light through the tube using fiber optics. That concept accommodates use of a very bright light source, but creates its own problems. Among other things an external light source is bulky and generally requires a plug in type power source rather than merely a battery.

It has additionally been suggested, although apparently not commercialized, to include a light bulb within the tube itself. For example, in U.S. Pat. No. 5,507,284 to Daneshvar (April 1996), an intubation tube includes a small battery powered light bulb 35. Daneshvar claims to place the bulb near the tip of the tube, but by that he means that the bulb should be positioned distal to the cuff, so that when the tube is properly inserted, the bulb is “in middle of the distance between the Cricoid cartilage and the upper notch of the sternum (the center bone of the chest).” (Spec. col. 11, line 66 through col. 12, line 13).

There are at least two significant problems with Daneshvar's devices. First, light bulbs suffer from a tradeoff between brightness and temperature. Sufficiently bright bulbs risk burning the patient. Second, Daneshvar uses the light to establish that the tube is properly inserted. Since the bulb is not at the tip portion of the tube, it has little or no usefulness in directing the tube during insertion.

What is needed is an endotracheal tube having a low temperature, built-in light source, which is positioned to provide improved assistance in guiding insertion of the tube.

### SUMMARY OF THE INVENTION

The present invention provides apparatus, systems and methods in which an endotracheal tube has a light-emitting diode or other low temperature, built-in light source forward of the cuff.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWING

FIG. 1 is a plan view of an endotracheal tube according to the present invention.

FIG. 2 is a plan view of an alternative endotracheal tube according to the present invention.

### DETAILED DESCRIPTION

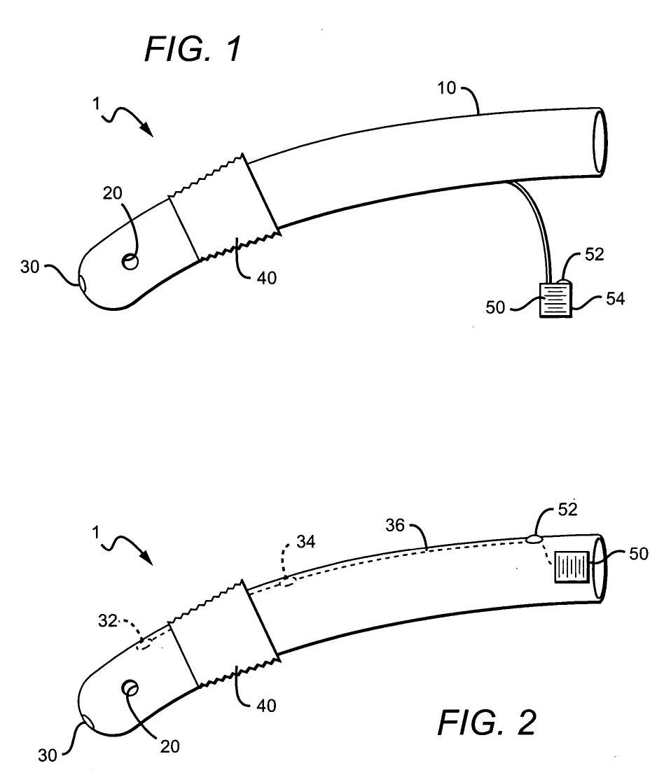

In FIG. 1 an endotracheal tube 1 generally has a tube body 10, an air hole 20, a light source 30, a cuff 40, and a power supply 50.

Tube body 10 can be manufactured from any suitable material, and can have any suitable dimensions. Indeed the present inventor contemplates use of all materials and dimensions previously used to manufacture endotracheal tube bodies, as well as their replacements. Preference, however, is more restricted. Any material overlying the light source 30 should be sufficiently transparent or translucent to pass a desirable intensity of visible light.

Air hole 20 and cuff 40 are similarly contemplated to conform to any suitable materials, positions, dimensions, and so forth. Pediatric devices, for example, would have significantly smaller dimensions than devices intended for adults.

In FIG. 1, the light source 30 is positioned near, but not at, the end of tube 1. The term “near” is defined herein as within 2 cm, and more preferably within 1 cm. Light source 30 is proximal to the cuff because it is closer to the air-hole end of tube 1 than the cuff 40.

Light source 30 is preferably a light-emitting diode (LED) or other solid state light emitter. The light emissions are preferably centered in the visible light range of about 400 nm to about 900 nm, and more preferably in the red region because of greater transmissibility through skin. A red LED is most preferred, but a white light emitting or other LED that includes some percentage of red light is also preferred.

In contrast to a typical tracheoscope, in which the emission from the light source is directed forwardly, at least some of the emission from the light source 30 should be directed normally to the tube, or at some other angle that would facilitate transdermal visualization.

The light source 30 can have a single emitter, or more advantageously can have multiple emitters centered at different colors, so that the physician or other user can alter the emitted color to maximize visibility through the patient's overlying tissue. Since solid state light emitters tend to use minimal power, the power supply 50 is typically a small button, AAA or other battery. Switch 52 controls voltage to the light source 30, and in its simplest embodiment can comprise an on-off switch. In more sophisticated embodiments switch 52 controls level of intensity, and/or color temperature. The battery 50 and switch 52 can be housed in an appropriate housing 54.

In FIG. 2 the light source 30 is positioned at the end of the tube 1. The battery 50 is disposed on or in the tube 1, and the switch 52 is attached to the tube 1. Optional second lights 32, 34 can be positioned anywhere on the tube 1. Wires 36 carry power from the battery 60 to the light source(s).

Thus, specific embodiments and applications of end-lighted endotracheal tubes have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps can be present, or utilized, or combined with other elements, components, or steps that are not expressly referenced.

What is claimed is:

1. An endotracheal tube having a proximal tip and a cuff, and a built-in light proximal to the cuff.

2. The tube of claim 1, wherein the tube has a concave curvature and the light is positioned on the concave curvature.

3. The tube of claim 1, wherein the light comprises a solid state emitter.

4. The tube of claim 3, wherein the light comprises a light emitting diode.

5. The tube of claim 3, further comprising a controller that modifies spectral distribution emitted from the light.

6. The tube of claim 1, further comprising a second light positioned to transilluminate an intubated patient.

7. The tube of claim 1, wherein the light is positioned at an end of the tube.

8. The tube of claim 1, wherein the light is positioned near an end of the tube.

9. The tube of claim 1, further comprising a power supply external to the tube.

10. The tube of claim 1, further comprising a power supply internal to the tube.

11. The tube of claim 1, further comprising an air hole, and the light is positioned proximal to the air hole.
